SEQUENCE LISTING (6 sequences)

SEQ ID NO: 1 - protein, 116 aa, Mus musculus
MEQRKGLAGLFLVISLLQGTVAQTNKAKNLVQVDGSRGDGSVLLTCGLTDKTIKWLKDGSIISPLNATKNTWNLGNNAKDPRGTYQCQGAKETSNPLQVYYRMCENCIELNIGTIS

SEQ ID NO: 2 - protein, 116 aa, Mus musculus; identical to SEQ ID NO: 1

SEQ ID NO: 3 - protein, 116 aa, Mus musculus; identical to SEQ ID NO: 1

SEQ ID NO: 4 - protein, 108 aa, Mus musculus
MRWNTFWGILCLSLLAVGTCQDDAENIEYKVSISGTSVELTCPLDSDENLKWEKNGQELPQKHDKHLVLQDFSEVEDSGYYVCYTPASNKNTYLYLKARVCEYCVEVD

SEQ ID NO: 5 - protein, 108 aa, Mus musculus
MRWNTFWGILCLSLLAVGTCQDDAENIEYKVSISGTSVELTCPLDSDENLKWEKNGQELPQKHDKHLVLQDFSEVEDSGYYVCYTPASKKNTYLYLKARVCEYCVEVD

SEQ ID NO: 6 - protein, 108 aa, Mus musculus; identical to SEQ ID NO: 5

(19) United States
(12) Patent Application Publication
(10) Pub. No.: US 2014/0141020 A1
(43) Pub. Date: May 22, 2014

(54) ANTI-CD3 THERAPIES

(71) Applicants: Diana Gil Pages, Rochester, MN (US); Adam G. Schrum, Rochester, MN (US)

(72) Inventors: Diana Gil Pages, Rochester, MN (US); Adam G. Schrum, Rochester, MN (US)

(73) Assignee: Mayo Foundation for Medical Education and Research, Rochester, MN (US)

(21) Appl. No.: 14/126,334

(22) PCT Filed: Jun. 5, 2012
(86) PCT No.: PCT/US2012/040919

§ 371 Date: Feb. 4, 2014

Related U.S. Application Data
(60) Provisional application No. 61/496,886, filed on Jun. 14, 2011.

(51) Int. Cl. A61K 39/395

(52) U.S. Cl. CPC A61K 39/39566
USPC 424/173.1; 530/389.6; 530/387.3

(57) ABSTRACT

This document provides methods and materials related to anti-CD3 therapies. For example, anti-CD3γε antibody preparations as well as methods and materials for using anti-CD3γε antibody preparations to reduce a T cell-mediated immune response within a mammal are provided.

### CROSS-REFERENCE TO RELATED APPLICATION

This application claims benefit of U.S. Provisional Application Ser. No. 61/496,886, filed Jun. 14, 2011. The disclosure of the prior application is considered part of (and is incorporated by reference in) the disclosure of this application.

### BACKGROUND

1. Technical Field

This document provides methods and materials related to anti-CD3 therapies. For example, this document provides anti-CD3γε antibody preparations as well as methods and materials for using anti-CD3γε antibody preparations to reduce a T cell-mediated immune response within a mammal.

2. Background Information

T cells can drive adaptive immune responses that can be pathogenic when directed against a body's own tissues and organs. In addition, T cells can be the main cellular component that drives acute organ rejection after transplantation. Different strategies for depleting/inactivating T cells have been pursued to treat patients either suffering from autoimmune diseases or requiring organ transplantation. While non-immunolglobulin drugs can be efficient to cause T cell cytotoxicity, they fail to be specific and they also can affect other cells. In an attempt to achieve cell specificity and cause T cell neutralization, immunoglobulin strategies using antibodies directed against a specific T cell molecule, CD3ε, were developed and have been used in an attempt to control acute organ rejection in transplanted patients. In addition, results have been obtained from clinical trials studying the efficacy of anti-CD3ε antibodies against several autoimmune diseases where T cells appear to be pathogenic.

Anti-CD3ε antibodies can neutralize T cell function mainly by causing the death of T cells, but can also result in the undesired activation of some T cells that escape the death. These residual T cells can secrete immunogenic cytokines that cause a set of deleterious symptoms, described as a cytokine release syndrome (CRS), which can include fever, chills, diarrhea, vomiting, respiratory problems, circulatory problems, and/or neurological problems.

### SUMMARY

This document provides methods and materials related to anti-CD3 therapies. For example, this document provides anti-CD3γε antibody preparations as well as methods and materials for using anti-CD3γε antibody preparations to reduce or block a T cell-mediated immune response within a mammal. In some cases, an anti-CD3γε antibody preparation provided herein can be used as an immunosuppressive agent to treat a mammal undergoing an organ transplant to reduce the likelihood of acute organ rejection. In some cases, an anti-CD3γε antibody preparation provided herein can be used as an immunosuppressive agent to treat an autoimmune disease (e.g., an autoimmune disease driven by T cell function) such as type I diabetes, multiple sclerosis, rheumatoid arthritis, Crohn's disease, or GVHD. In some cases, an anti-CD3γε antibody preparation provided herein can be used to treat a mammal suffering from cancer such as T cell lymphoma.

As described herein, anti-CD3γε antibody preparations can be used to induce immunosuppression in a manner that does not induce a cytokine release syndrome. In some cases, an anti-CD3γε antibody preparation provided herein can be used to induce immunosuppression in a manner such that the antibodies are not internalized and do not crosslink TCR/CD3 in a manner that causes a full T cell response or T cell proliferation. In some cases, an anti-CD3γε antibody preparation provided herein can be used to cause the death of T cells by, for example, apoptosis without provoking either T cell division or adverse cytokine production (e.g., production of elevated levels of IL-1β, IL-2, IL-4, or TNF-α). For example, an anti-CD3γε antibody preparation provided herein can be capable of programming or triggering apoptosis in otherwise unstimulated T cells so the T cells are depleted without causing adverse side effects like hypothermia, diarrhea, hypoglycemia, morbidity, and/or mortality. In some cases, an anti-CD3γε antibody preparation provided herein can be used to induce immunosuppression in a manner that reduces the severity and/or allows recuperation (e.g., full recuperation) from an autoimmune disease driven by T cell function such as multiple sclerosis.

In general, one aspect of this document features an anti-CD3γε antibody preparation comprising, or consisting essentially of, Fab fragments of an anti-CD3γε antibody. The Fab fragments can be Fab fragments of an anti-human CD3γε antibody. The Fab fragments can be Fab fragments of a humanized anti-human CD3γε antibody. The Fab fragments can be Fab fragments of a fully human anti-human CD3γε antibody. Administration of the preparation to a mammal can induce T cell death within the mammal. Administration of the preparation to a mammal can induce T cell death within the mammal with no detectable T cell division. Administration of the preparation to a mammal can induce T cell death within the mammal with no detectable increases in IL-1β production. Administration of the preparation to a mammal can induce T cell death within the mammal with no detectable increases in IL-2 production. Administration of the preparation to a mammal can induce T cell death within the mammal with no detectable increases in IL-4 production. Administration of the preparation to a mammal can induce T cell death within the mammal with no detectable increases in TNF-α production. Administration of the preparation to a mammal can induce T cell death within the mammal with no detectable increases in IL-1β, IL-2, IL-4, and TNF-α production.

In another aspect, this document features a method for reducing the likelihood of transplant rejection in a mammal. The method comprises, or consists essentially of, administering an anti-CD3γε antibody preparation comprising Fab fragments of an anti-CD3γε antibody to the mammal under conditions wherein at least a portion of T cells present within the mammal are killed with no detectable increase in IL-1β, IL-2, IL-4, or TNF-α production. The mammal can be a human, and the Fab fragments can be Fab fragments of an anti-human CD3γε antibody. The Fab fragments can be Fab fragments of a humanized anti-human CD3γε antibody. The Fab fragments can be Fab fragments of a fully human anti-human CD3γε antibody.

In another aspect, this document features a method for treating a mammal having an autoimmune condition. The method comprises, or consists essentially of, administering an anti-CD3γε antibody preparation comprising Fab fragments of an anti-CD3γε antibody to the mammal under conditions wherein at least a portion of T cells present within the mammal are killed with no detectable increase in IL-1β, IL-2, IL-4, or TNF-α production. The mammal can be a human, and the Fab fragments can be Fab fragments of an anti-human CD3γε antibody. The Fab fragments can be Fab fragments of a humanized anti-human CD3γε antibody. The Fab fragments can be Fab fragments of a fully human anti-human CD3γε antibody. The autoimmune condition can be diabetes or multiple sclerosis.

In another aspect, this document features a method for treating a mammal having cancer. The method comprises, or consists essentially of, administering an anti-CD3γε antibody preparation comprising Fab fragments of an anti-CD3γε antibody to the mammal under conditions wherein at least a portion of T cells present within the mammal are killed with no detectable increase in IL-1β, IL-2, IL-4, or TNF-α production. The mammal can be a human, and the Fab fragments can be Fab fragments of an anti-human CD3γε antibody. The Fab fragments can be Fab fragments of a humanized anti-human CD3γε antibody. The Fab fragments can be Fab fragments of a fully human anti-human CD3γε antibody. The cancer can be T cell lymphoma.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1. Western blot quality controls for 7D6 Fab preparation. Titrated amounts of 7D6 mAb or Fab preparations were analyzed by SDS-PAGE and Western blot with anti-mouse IgG. The Fab was about 50 kDa. The smaller molecular weight Ig species in the Fab lanes originated from intact Fab that falls apart during boiling and preparation for Western blot prep (as confirmed by SEC analysis). 7D6 digestion appeared free of undigested 7D6 mAb at the level of detection of the assay. Thus, a ratio of 7D6 Fab/mAb was established as being ≦1 μg/ng in the preparation.

FIG. 2. Binding and stimulatory capacity of 7D6 Fab on T cells. (A) Thy1.2-gated B6 splenocytes stained positively with 7D6 mAb and Fab, but not with irrelevant mouse IgG, as detected via anti-mouse IgG-FITC secondary reagent. (B) After 24 hours of culture at 37° C., 7D6 mAb, but not 7D6 Fab or irrelevant mouse IgG treatment, induced surface TCR downregulation. (C) After 24 hours of culture at 37° C., 7D6 mAb and Fab, but not irrelevant mouse IgG, induced CD69 upregulation. (D) B6 splenocytes were treated as indicated, lysed, and analyzed by the CD3-PD assay, detecting a positive induction of open-CD3 conformation by 7D6 Fab.

FIG. 3. 7D6 Fab does not block pMHC:TCR binding. Splenocytes from OT-I TCR transgenic mice were stained on ice with Fabs as indicated for 40 minutes. Next, without washing away excess Fab, cells were stained with titrated amounts of PE-labeled K^{b}/OVA MHC tetramers. Whereas a 2C11-Fab blocked tetramer binding, 7D6-Fab did not.

FIG. 4. 7D6 Fab did not block T cell response to antigens. OT-I CTLs, generated in a four day co-culture of OT-I splenocytes with OVA loaded DCs, were mixed with OVA pulsed EL4 target cells in the presence of mouse IgG control or 7D6 Fab. After 6 hours of incubation at 37° C., the specific killing of EL-4 target cells was monitored by a flow cytometry assay.

FIG. 5. 7D6 Fab induced death of T cells and Fas up-regulation independently and dominant to IL2 and antigenic TCR stimulation. OT-I splenocytes were co-cultured with bone marrow derived DCs that were treated with LPS and subsequently loaded with either no peptide or pOVA (SIINFEKL peptide, specific for the OT-I TCR). Co-cultures were plated in the absence or presence of a saturating amount of IL2 and in the absence or presence of 7D6 Fab. After four days, co-cultures were stained for Thy1.2 and Fas surface expression. PI was added to the staining procedure to label dead cells, and samples were analyzed by flow cytometry. (A) Counts of T cells alive determined by analysis of forward and size scatter in addition to PI exclusion. (B) Percent T cells alive that express Fas.

FIG. 6. 7D6 Fab induced T cell death without causing T cell division. OT-I splenocytes co-cultured with DCs from FIG. 5 were CFSE labeled prior to plating. After four days, the CFSE profile of PI⁻ Thy1.2⁺ cells was monitored by flow cytometry to account for T cell division induced in the co-cultures. CFSE profiles of co-cultures in the absence (A) or presence (B) of IL2 are shown.

FIG. 7. Isolated engagement of CD3γε dimers, but not co-engagement of CD3γε/εδ dimers, by anti-CD3 Fabs induced T cell death without T cell division. CFSE labeled OT-I splenocytes were co-cultured in IL2 with DCs as in FIG. 5 either in the presence of no IgG, 7D6 Fab, or 2C11 Fab. (A) After four days, co-cultures were stained for Thy1.2, and counts of T cells alive were determined by analysis of forward and size scatter in addition to PI cell exclusion. (B) CFSE profile of PI⁻ Thy1.2⁺ cells was monitored as well to account for T cell division in the co-cultures.

FIG. 8. Isolated engagement of CD3γε dimers by 7D6 Fab was unique in its ability to induce T cell death. OT-I splenocytes were co-cultured in IL2 with DCs as in FIG. 5, either in the presence of no IgG, control mouse IgG mAb and Fab, or the anti-CD3γε 7D6 and 17A2 Fabs and mAbs. After four days, co-cultures were stained for Thy1.2, and counts of T cells alive were determined by analysis of forward and size scatter in addition to PI cell exclusion.

FIG. 9. 7D6 Fab was capable of killing T cell blasts through binding to the CD3γε dimer. (A) B6 wild type and CD3γ^{-/-} T cell blasts derived from stimulation of splenocytes for four days with plate bound anti-CD3 were rested for one day and then incubated for 24 hours with either soluble mouse IgG, 7D6 Fab, or 7D6 mAb. Living cells counts were obtained analyzing forward and size scatter and PI exclusion by flow cytometry, and a percentage of survival of T cell blasts in the presence of 7D6 Fab and mAb was calculated by comparison with the mouse IgG condition. (B) T cell blasts generated from splenocytes of B6 and the LPJ and 129/Sv strains that do not stain with 7D6 due to allelic variation in CD3ε were stimulated for 24 hours as in (A), and a percentage survival of T cells blasts in the presence of 7D6 Fab and mAb was calculated.

FIG. 10. Epitope recognized by 7D6 Fab in mouse CD3γε dimer. mRNA coding for CD3γ (A) and CD3ε (B) from the three mouse strains were sequenced and aligned to identify allelic variations in the sequence of amino acids. One change in the residue at position 91 in the B6 sequence for CD3ε was found. This indicates that the epitope recognized by 7D6 in B6 may include residue 91. (C) Structure of CD3γε extra-cellular domain adapted from Sun et al. (Cell, 105:915-923 (2001)). The arrow locates residue 91 in CD3ε. 7D6 epitope may be shaped by residues included in the F/G loops in CD3γ and CD3ε.

FIG. 11. 7D6 Fab induced apoptosis of activated T cells. (A) B6 T cell blasts derived from stimulation of splenocytes for four days with plate bound anti-CD3 were rested for one day and then incubated with either soluble mouse IgG, 7D6 Fab, or 7D6 mAb. At the time point indicated, living CD8⁺ and CD4⁺ T cell blasts were monitored for Annexin-V staining by flow cytometry. (B) B6 T cell blasts rested for one day were stimulated for 24 hours with either soluble mouse IgG, 7D6 Fab, or 7D6 mAb, in the presence or absence of a Fas/FasL blocking cocktail. The percentage of survival of T cells blasts was calculated relative to the mouse IgG conditions as in FIG. 9.

FIG. 12. 7D6 Fab molecules in solution form non-covalent oligomers (7D6-Oligo-Fabs) that correlate with the T cell killing activity of 7D6 Fab. (A) SEC absorbance profile of 7D6 mAb, 7D6 F(ab′)₂, and 7D6 Fab freshly prepared (7D6 Fab fresh) when fractionated over a Superose-200 column (S-200) equilibrated in PBS. The expected molecular weight for each 7D6 species tested is in brackets. (B) 7D6 Fab fresh fractions collected in (A) were run in SDS-PAGE non-reducing conditions to detect 7D6 by anti-mouse western blotting. The peak of maximum 7D6 material found in fraction 10 matches the absorbance profile of 7D6 Fab fresh in (A) and has a molecular weight between 37 and 50 KDa, as expected for a Fab fragment. (C) Killing activity test against T cell blasts performed as in FIG. 9 with the 14 fractions of 7D6 Fab fresh collected in (A). The peak of maximum killing activity is in fraction 9. (D) SEC absorbance profile of 7D6 Fab 40 days after its preparation when fractionated over an S-200 equilibrated in PBS. The profile reflects the presence of a second molecular species that matches the 7D6 F(ab′)₂ profile shown in (A). This indicates a molecular size of about 100 KDa in native conditions for the new species found 40 days after preparing 7D6 Fab. (E) 7D6 Fab+40 days fractions collected in (D) were run in SDS-PAGE non-reducing conditions to detect 7D6 by anti-mouse western blotting. The peak of 7D6 material appears in fractions 9 and 10, matching the peaks observed in (D). (F) Killing activity test against T cell blasts performed as in FIG. 9 with the 14 fractions collected in (D) for the 7D6 Fab+40 days. Note the peak of maximum killing activity is in fraction 9.

FIG. 13. Intraperitoneal (i.p.) injection of 7D6-Oligo-Fab into B6 mice induced T cell depletion from peripheral blood. Panel A is a diagram of the injection scheme applied on B6 mice for panels B and C. (B) Three B6 mice per experimental group were injected with the indicated doses of 7D6 mAb and Fab, and PBLs were isolated from blood. The expression of B2.20 and Thy1.2 surface markers on the PBLs was monitored by flow cytometry, and a T to B cell ratio was calculated in each sample based on the results of the stainings. (C) Three B6 mice per experimental group were injected with the indicated doses of either 7D6 Fab or mAb. T/B cell ratio was calculated as in Panel B.

FIG. 14. 7D6-Oligo-Fab did not cause cytokine release syndrome (CRS). Three B6 mice per experimental group were injected i.v. with 20 μg of the non-specific mouse IgG Fab, 7D6-Oligo-Fab, 7D6 mAb, or 2C11 mAb. Two hours later, blood samples were collected, and sera were prepared and analyzed for the specified cytokine concentrations by ELISA. Mice also were monitored for piloerection, diarrhea, and hypoglycimia, testing negative for all related measurements.

FIG. 15. 7D6 Ab induced transitory T cell depletion from peripheral blood in B6 mice. Three B6 mice per experimental group were injected retro-orbitally with the indicated doses of either mouse IgG control or different 7D6 Abs. 24 and 48 hours post-injection, mice were bled, and PBLs were isolated. Surface expression of the B cell marker B2.20 and the T cell marker Thy1.2 was monitored in the PBLs by flow cytometry. A T to B cell ratio was calculated in each sample based on the results of the B2.20 and Thy1.2 stainings.

FIG. 16. Repeated injections of 7D6-Oligo-Fab achieved a sustained depletion of T cells from peripheral blood in B6 mice. (A) Three B6 mice per experimental group were injected with the indicated doses of 7D6-Oligo-Fab. Mice receiving a 20 μg dose were re-injected on day 2 and 4 after their first injection. At the indicated time points, mice were bled, and PBLs were isolated from blood. The expression of B2.20 and Thy1.2 surface markers on the PBLs was monitored by flow cytometry, and a T to B cell ratio was calculated. Panel B contains results of an extended kinetic monitoring T/B cell ratio in PBLs for the mice treated with 20 μg 7D6-Oligo-Fab.

FIG. 17. Injection of 7D6-Oligo-Fab resulted in decreased Tot1.1-tumor burden in B6 mice. The Tot1.1 cell line was derived upon extensive in vitro culture of a spontaneous T cell lymphoma that occurs in a B6 OT-I mouse colony. Five cage-mate B6 mice were injected i.v. with 0.5×10⁶ Tot1.1 tumor cells. Six days later, two mice were injected i.p. with PBS, while three mice were injected i.p. with 20 μg of mono-7D6-Fab. After 17 days, mice injected with PBS showed significant signs of morbidity, but those injected with mono-7D6-Fab did not. All mice were sacrificed on day 17, and putative tumor-containing nodules were harvested from livers. Each putative tumor-containing nodule was placed in its own tissue culture well for one week. Nodules were verified as tumor-positive if Tot1.1 cells grew from them in culture.

FIG. 18. Pre-treatment with 7D6-Oligo-Fab protects from EAE. (A) On days −5, −3, and −1, B6 mice were injected intravenously with either 20 μg M IgG Fab or 7D6-Oligo-Fab (5 mice/condition). 24 hours post-Fab injection, mice were bled, and T/B cell ratios were determined. (B) On day 0, all mice underwent a protocol to induce EAE: (i) subcutaneous immunization in both flanks with 100 μg of MOG peptide (amino acid residues 35-55) emulsified in CFA containing M. tuberculosis H37Ra (400 μg/mouse): (ii) i.p. injection of Pertussis toxin (100 ng/mouse) on days 0 and 2. Mice were observed daily for symptoms of EAE and clinically scored as follows: 0, normal; 1, loss of tail tone; 2, hind limb weakness; 3, hind limb paralysis; 4, hind limb paralysis and forelimb paralysis or weakness; and 5, moribund, sufficient for mouse sacrifice. By day 47, all mice from the 7D6-Oligo-Fab experimental group were recovered from EAE and scored as 0, and remained symptom-free for as long as they were monitored, until at least day 77.

### DETAILED DESCRIPTION

This document provides methods and materials related to anti-CD3γε antibody preparations. For example, this document provides anti-CD3γε antibody preparations, methods for making anti-CD3γε antibody preparations, and methods for using anti-CD3γε antibody preparations as an immunosuppressive agent to reduce the likelihood of organ rejection, to treat an autoimmune disease or conditions, and/or to treat cancers such as T cell lymphomas. In some cases, an anti-CD3γε antibody preparation provided herein can bind to a CD3γε dimer with little or no detectable binding to a CD3ε polypeptide not in the form of a CD3γε dimer and with little or no detectable binding to a CD3γ polypeptide not in the form of a CD3γε dimer. For example, an anti-CD3γε antibody preparation provided herein can bind to a human CD3γε dimer with little or no detectable binding to a human CD3ε polypeptide not in the form of a CD3γε dimer and with little or no detectable binding to a human CD3γ polypeptide not in the form of a CD3γε dimer. An example of an antibody having the ability to bind to a CD3γε dimer with little or no detectable binding to a CD3εpolypeptide not in the form of a CD3γε dimer and with little or no detectable binding to a CD3γ polypeptide not in the form of a CD3γε dimer includes, without limitation, the 7D6 antibody described elsewhere (Van Snick et al., Eu. J. Immunol., 21:1703-1709 (1991)).

The term “antibody” as used herein refers to intact antibodies as well as antibody fragments that retain some ability to bind an epitope. Such fragments include, without limitation, Fab, F(ab′)2, and Fv antibody fragments. The term “epitope” refers to an antigenic determinant on an antigen to which the paratope of an antibody binds. Epitopic determinants usually consist of chemically active surface groupings of molecules (e.g., amino acid or sugar residues) and usually have specific three dimensional structural characteristics as well as specific charge characteristics.

The antibodies provided herein can be any antibody (e.g., a monoclonal antibody) having binding affinity (e.g., specific binding affinity) for a CD3γε dimer with little or no detectable binding to a CD3ε polypeptide not in the form of a CD3γε dimer or a CD3γ polypeptide not in the form of a CD3γε dimer. For example, an anti-CD3γε antibody preparation provided herein can be a preparation of Fab fragments having the ability to bind to a CD3γε dimer with little or no detectable binding to a CD3ε polypeptide not in the form of a CD3γε dimer and with little or no detectable binding to a CD3γ polypeptide not in the form of a CD3γε dimer. In some cases, the Fab fragments of an anti-CD3γε antibody preparation provided herein can form non-covalently associated oligomers. In some cases, the non-covalent oligomers can be composed of two, three, or four Fab fragments of anti-CD3γε dimer antibodies. For example, two, three, or four Fab fragments of humanized or fully-human anti-human CD3γε dimer antibodies can be non-covalently associated with each other to form an oligomer of Fab fragments. Any appropriate method can be used to produce Fab fragments from intact antibodies. For example, standard papain digestion methods can be used to make an Fab antibody preparation. Any appropriate method can be used to make an anti-CD3γε antibody preparation of Fab fragments in the form of oligomers. For example, an Fab antibody preparation can be incubated at 4° C. for at least three weeks to allow formation of the non-covalent oligomers.

In some cases, an anti-CD3γε antibody preparation provided herein can be a preparation of Fab fragments of humanized or fully-human anti-human CD3γε dimer antibodies that are in the form of non-covalently associated oligomers having between two and four Fab fragments per oligomer.

In some cases, an anti-CD3γε antibody preparation provided herein (e.g., an anti-CD3γε antibody preparation containing Fab fragments of a humanized anti-CD3γε antibody or an oligomers of Fab fragments of a humanized anti-CD3γε antibody) can induce little, if any, detectable levels of IL-1β, IL-2, IL-4, and/or TNF-α when administered to a mammal (e.g., a human). For example, an anti-CD3γε antibody preparation provided herein (e.g., an anti-CD3γε antibody preparation containing Fab fragments of a humanized anti-CD3γε antibody or an oligomers of Fab fragments of a humanized anti-CD3γε antibody) can, when therapeutically administered to a human, lack the ability to induce secretion of levels of IL-1β, IL-2, IL-4, and/or TNF-α that are detectable (e.g., detectable using standard ELISA techniques).

The anti-CD3γε antibody preparations provided herein can be used as an immunosuppressive agent to reduce the likelihood of organ rejection (e.g., kidney, liver, heart, lung, pancreas, or islets rejection). For example, an anti-CD3γε antibody preparation provided herein such as a preparation of Fab fragments of humanized or fully-human anti-human CD3γε dimer antibodies can be administered to human transplant patients using techniques similar to those described elsewhere (U.S. Pat. Nos. 6,491,916 and 6,406,696).

In some cases, the anti-CD3γε antibody preparations provided herein can be used as an immunosuppressive agent to treat an autoimmune disease or condition (e.g., diabetes, multiple sclerosis, rheumatoid arthritis, Crohn's disease, or GVHD). For example, an anti-CD3γε antibody preparation provided herein such as a preparation of Fab fragments of humanized or fully-human anti-human CD3γε dimer antibodies can be administered to a human patient having an autoimmune disease or condition using techniques similar to those described elsewhere (U.S. Patent Application Publication No. 2008/0095766).

In some cases, the anti-CD3γε antibody preparations provided herein can be used as an immunosuppressive agent to treat T cell cancers such as T cell lymphomas. For example, an anti-CD3γε antibody preparation provided herein such as a preparation of Fab fragments of humanized or fully-human anti-human CD3γε dimer antibodies can be administered to a human cancer patient using techniques similar to those described elsewhere (U.S. Pat. No. 6,113,901).

Antibodies provided herein can be prepared using any method. For example, a sample containing a CD3γε dimer (e.g., a human CD3γε dimer or a chimeric mouse/human CD3γε dimer) can be used as an immunogen to elicit an immune response in an animal such that specific antibodies are produced. The immunogen used to immunize an animal can be chemically synthesized or derived from translated cDNA. In some cases, cells (e.g., mouse T cells) transfected to express a CD3γε dimer (e.g., a human CD3γε dimer or a chimeric mouse/human CD3γε dimer) can be used as an immunogen. In some cases, the immunogen can be conjugated to a carrier polypeptide, if desired. Commonly used carriers that are chemically coupled to an immunizing polypeptide include, without limitation, keyhole limpet hemocyanin (KLH), thyroglobulin, bovine serum albumin (BSA), and tetanus toxoid.

The preparation of polyclonal antibodies is well-known to those skilled in the art. See, e.g., Green et al., Production of Polyclonal Antisera, in IMMUNOCHEMICAL PROTOCOLS (Manson, ed.), pages 1 5 (Humana Press 1992) and Coligan et al., Production of Polyclonal Antisera in Rabbits, Rats, Mice and Hamsters, in CURRENT PROTOCOLS IN IMMUNOLOGY, section 2.4.1 (1992). In addition, those of skill in the art will know of various techniques common in the immunology arts for purification and concentration of polyclonal antibodies, as well as monoclonal antibodies (Coligan, et al., Unit 9, Current Protocols in Immunology, Wiley Interscience, 1994). The preparation of monoclonal antibodies also is well-known to those skilled in the art. See, e.g., Kohler & Milstein, Nature 256:495 (1975); Coligan et al., sections 2.5.1 2.6.7; and Harlow et al., ANTIBODIES: A LABORATORY MANUAL, page 726 (Cold Spring Harbor Pub. 1988). Briefly, monoclonal antibodies can be obtained by injecting mice with a composition comprising an antigen, verifying the presence of antibody production by analyzing a serum sample, removing the spleen to obtain B lymphocytes, fusing the B lymphocytes with myeloma cells to produce hybridomas, cloning the hybridomas, selecting positive clones that produce antibodies to the antigen, and isolating the antibodies from the hybridoma cultures. Monoclonal antibodies can be isolated and purified from hybridoma cultures by a variety of well established techniques. Such isolation techniques include affinity chromatography with Protein A Sepharose, size exclusion chromatography, and ion exchange chromatography. See, e.g., Coligan et al., sections 2.7.1 2.7.12 and sections 2.9.1 2.9.3; Barnes et al., Purification of Immunoglobulin G (IgG), in METHODS IN MOLECULAR BIOLOGY, VOL. 10, pages 79 104 (Humana Press 1992).

In addition, methods of in vitro and in vivo multiplication of monoclonal antibodies are well known to those skilled in the art. Multiplication in vitro can be carried out in suitable culture media such as Dulbecco's Modified Eagle Medium or RPMI 1640 medium, optionally replenished by mammalian serum such as fetal calf serum, or trace elements and growth sustaining supplements such as normal mouse peritoneal exudate cells, spleen cells, and bone marrow macrophages. Production in vitro provides relatively pure antibody preparations and allows scale up to yield large amounts of the desired antibodies. Large scale hybridoma cultivation can be carried out by homogenous suspension culture in an airlift reactor, in a continuous stirrer reactor, or in immobilized or entrapped cell culture. Multiplication in vivo may be carried out by injecting cell clones into mammals histocompatible with the parent cells (e.g., osyngeneic mice) to cause growth of antibody producing tumors. Optionally, the animals are primed with a hydrocarbon, especially oils such as pristane (tetramethylpentadecane) prior to injection. After one to three weeks, the desired monoclonal antibody is recovered from the body fluid of the animal.

In some cases, the antibodies provided herein can be made using non-human primates. General techniques for raising therapeutically useful antibodies in baboons can be found, for example, in Goldenberg et al., International Patent Publication WO 91/11465 (1991) and Losman et al., Int. J. Cancer, 46:310 (1990).

In some cases, the antibodies can be humanized monoclonal antibodies. Humanized monoclonal antibodies can be produced by transferring mouse complementarity determining regions (CDRs) from heavy and light variable chains of the mouse immunoglobulin into a human variable domain, and then substituting human residues in the framework regions of the murine counterparts. The use of antibody components derived from humanized monoclonal antibodies obviates potential problems associated with the immunogenicity of murine constant regions when treating humans. General techniques for cloning murine immunoglobulin variable domains are described, for example, by Orlandi et al., Proc. Nat'l. Acad. Sci. USA 86:3833 (1989). Techniques for producing humanized monoclonal antibodies are described, for example, by Jones et al., Nature 321:522 (1986); Riechmann et al., Nature 332:323 (1988); Verhoeyen et al., Science 239:1534 (1988); Carter et al., Proc. Nat'l. Acad. Sci. USA 89:4285 (1992); and Sandhu, Crit. Rev. Biotech. 12:437 (1992); Singer et al., J. Immunol. 150:2844 (1993). In some cases, humanization such as super humanization can be used as described elsewhere (Hwang et al., Methods, 36:35-42 (2005)). In some cases, SDR grafting (Kashmiri et al., Methods, 36:25-34 (2005)), human string content optimization (Lazar et al., Mol. Immunol., 44:1986-1998 (2007)), framework shuffling (Dall'Acqua et al., Methods, 36:43-60 (2005); and Damschroder et al., Mol. Immunol., 44:3049-3060 (2007)), and phage display approaches (Rosok et al., J Biol. Chem., 271:22611-22618 (1996); Radar et al., Proc. Natl. Acad. Sci. USA, 95:8910-8915 (1998); and Huse et al., Science, 246:1275-1281 (1989)) can be used to obtain anti-CD3γε antibody preparations. In some cases, fully human antibodies can be generated from recombinant human antibody library screening techniques as described elsewhere (Griffiths et al., EMBO J., 13:3245-3260 (1994); and Knappik et al., J. Mol. Biol., 296:57-86 (2000)).

Antibodies provided herein can be derived from human antibody fragments isolated from a combinatorial immunoglobulin library. See, for example, Barbas et al., METHODS: A COMPANION TO METHODS IN ENZYMOLOGY, VOL. 2, page 119 (1991) and Winter et al., Ann. Rev. Immunol. 12: 433 (1994). Cloning and expression vectors that are useful for producing a human immunoglobulin phage library can be obtained, for example, from STRATAGENE Cloning Systems (La Jolla, Calif.).

In addition, antibodies provided herein can be derived from a human monoclonal antibody. Such antibodies can be obtained from transgenic mice that have been “engineered” to produce specific human antibodies in response to antigenic challenge. In this technique, elements of the human heavy and light chain loci are introduced into strains of mice derived from embryonic stem cell lines that contain targeted disruptions of the endogenous heavy and light chain loci. The transgenic mice can synthesize human antibodies specific for human antigens and can be used to produce human antibody secreting hybridomas. Methods for obtaining human antibodies from transgenic mice are described by Green et al. (Nature Genet., 7:13 (1994)), Lonberg et al. (Nature, 368:856 (1994)), and Taylor et al. (Int. Immunol., 6:579 (1994)).

Antibody fragments can be prepared by proteolytic hydrolysis of an intact antibody or by the expression of a nucleic acid encoding the fragment. Antibody fragments can be obtained by pepsin or papain digestion of intact antibodies by conventional methods. For example, Fab fragments can be produced by enzymatic cleavage of antibodies with papain. In some cases, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. In some cases, an enzymatic cleavage using pepsin can be used to produce two monovalent Fab′ fragments and an Fc fragment directly. These methods are described, for example, by Goldenberg (U.S. Pat. Nos. 4,036,945 and 4,331,647). See also Nisonhoff et al., Arch. Biochem. Biophys. 89:230 (1960); Porter, Biochem. J. 73:119 (1959); Edelman et al., METHODS IN ENZYMOLOGY, VOL. 1, page 422 (Academic Press 1967); and Coligan et al. at sections 2.8.1 2.8.10 and 2.10.1 2.10.4.

Other methods of cleaving antibodies, such as separation of heavy chains to form monovalent light heavy chain fragments, further cleavage of fragments, or other enzymatic, chemical, or genetic techniques may also be used provided the fragments retain some ability to bind (e.g., selectively bind) its epitope.

The antibodies provided herein can be substantially pure. The term “substantially pure” as used herein with reference to an antibody means the antibody is substantially free of other polypeptides, lipids, carbohydrates, and nucleic acid with which it is naturally associated. Thus, a substantially pure antibody is any antibody that is removed from its natural environment and is at least 60 percent pure. A substantially pure antibody can be at least about 65, 70, 75, 80, 85, 90, 95, or 99 percent pure.

The invention will be further described in the following examples, which do not limit the scope of the invention described in the claims.

### Examples

### Example 1

### Effects of Anti-CD3γε Dimer Antibodies

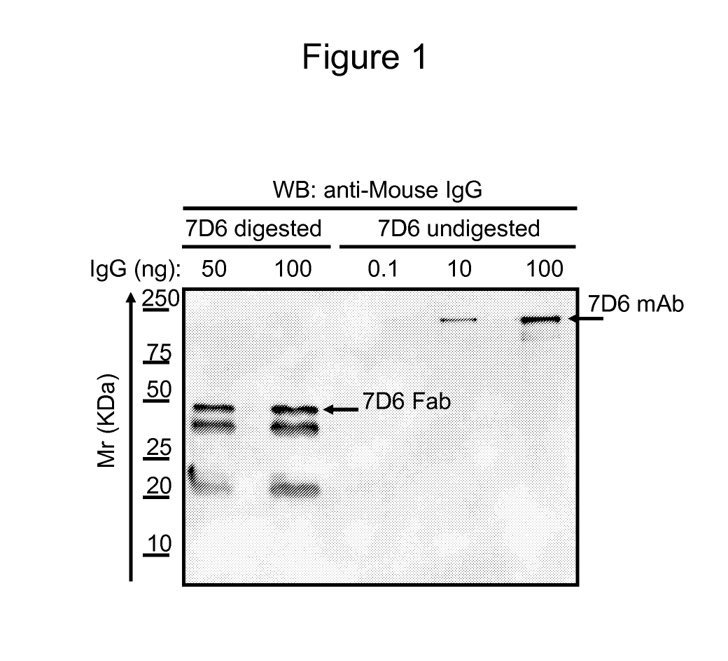

The monoclonal 7D6 antibody was generated by injecting T cells from a B6 mouse into 129/Sv mice as described elsewhere (Coulie et al., Eur. J. Immunol., 21: 1703-1709 (1991)). Fab fragments were made by digesting purified 7D6 mAb with papain during 24 hours at 37° C. FIG. 1 demonstrates Western blot quality controls for 7D6 Fab preparations. Titrated amounts of 7D6 mAb or Fab preparations were analyzed by SDS-PAGE and Western blot with anti-mouse IgG. The Fab was about 50 kDa. The smaller molecular weight Ig species in the Fab lanes originated from intact Fab that falls apart during boiling and preparation for Western blot prep (as confirmed by Size Exclusion Chromatography (SEC) analysis). 7D6 digestion appeared free of undigested 7D6 mAb at the level of detection of the assay. Thus, a ratio of 7D6 Fab/mAb was established as being ≦1 μg/ng in the preparation.

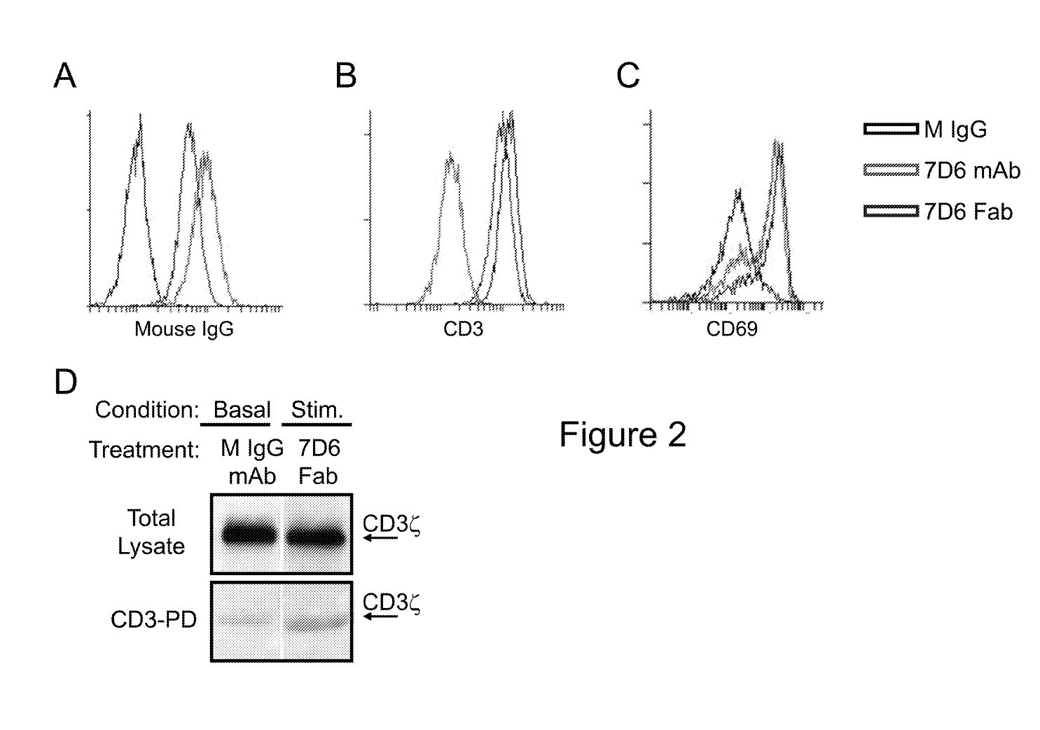

Next, the binding and stimulatory capacity of 7D6 Fab on T cells was tested. In FIG. 2A, Thy1.2-gated B6 splenocytes stained positively with 7D6 mAb and Fab, but not with irrelevant mouse IgG, as detected via anti-mouse IgG-FITC secondary reagent. After 24 hours of culture at 37° C., 7D6 mAb, but not 7D6 Fab or irrelevant mouse IgG treatment, induced surface TCR downregulation as shown in FIG. 2B. After 24 hours of culture at 37° C., 7D6 mAb and Fab, but not irrelevant mouse IgG, induced CD69 upregulation (FIG. 2C). For FIG. 2D, B6 splenocytes treated as indicated, lysed, and analyzed by the CD3-Pull Down (PD) assay undergo induction of open-CD3 conformation when treated with 7D6 Fab. These experiments demonstrate that 7D6 Fab binds to T cells and induces CD3 conformational change and CD69 up-regulation but fails to produce TCR/CD3 internalization.

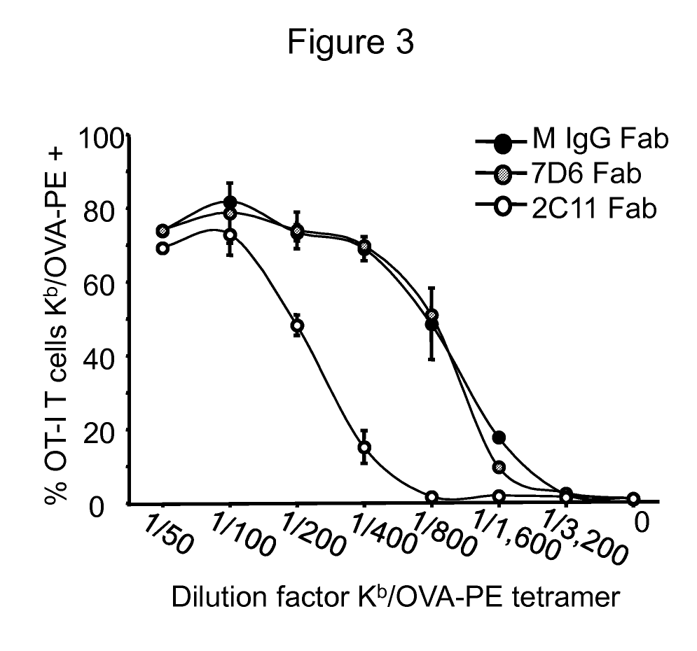

The effect of 7D6 Fab binding to T cells on their ability to recognize specific antigens through the TCR was tested. In FIG. 3, splenocytes from OT-I TCR transgenic mice were stained on ice with 7D6 or 2C11 Fabs as indicated for 40 minutes. Next, without washing away excess Fabs, cells were stained with titrated amounts of PE-labeled K^{b}/OVA MHC tetramers. The data demonstrates that whereas a 2C11-Fab blocked tetramer binding, 7D6 Fab did not.

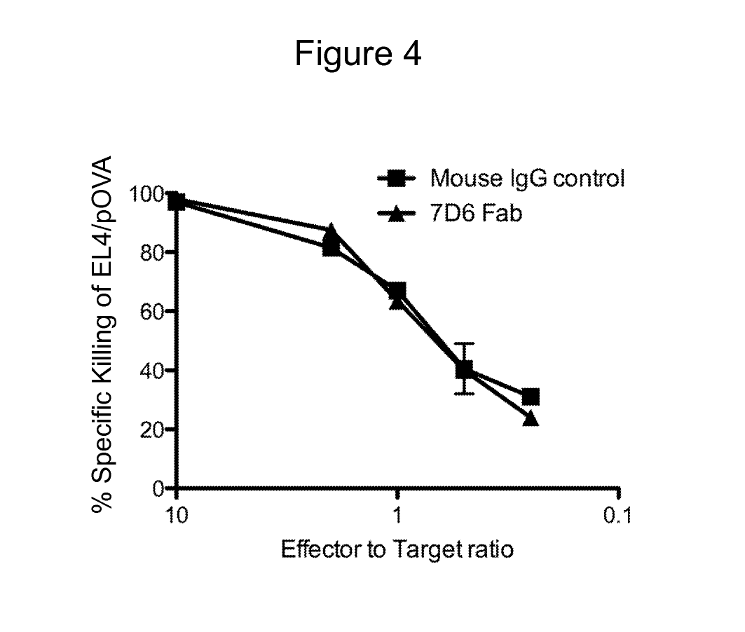

In addition, the capacity of 7D6 Fab to block T cell response to antigens was also tested. In FIG. 4, OT-I CTLs, generated in a four day co-culture of OT-I splenocytes with OVA loaded DCs, were mixed with OVA pulsed EL4 target cells in the presence of mouse IgG control or 7D6 Fab. After 6 hours of incubation at 37° C., the specific killing of EL-4 target cells was monitored by a flow cytometry assay. The killing of target cells loaded with the specific antigen was not inhibited by 7D6 Fab treatment, suggesting that 7D6 Fab binding to T cells does not block their capacity to respond to antigens.

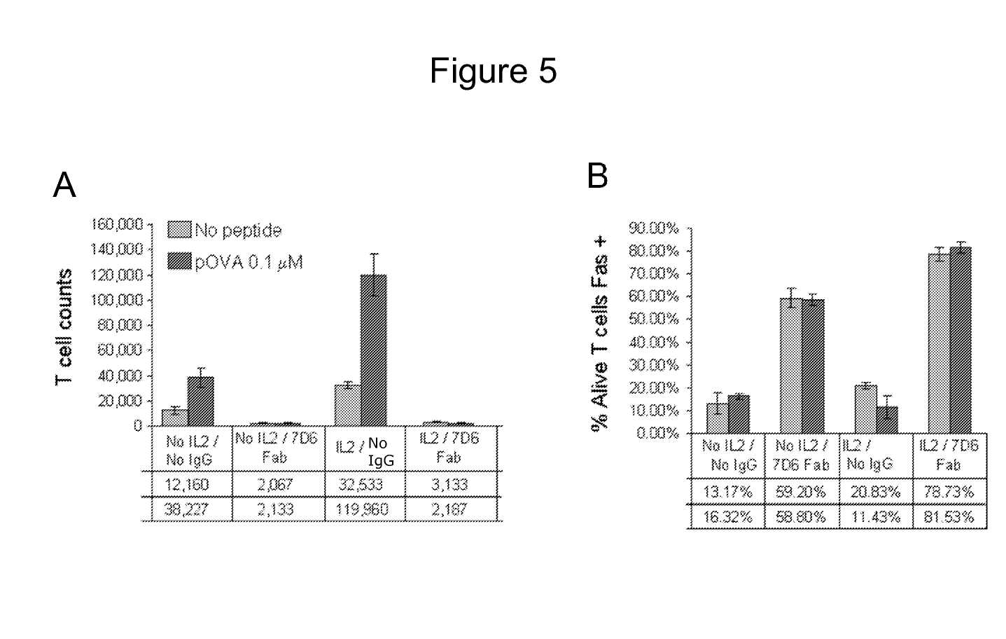

Separately, the effect of 7D6 Fab on T cell viability in different stimulatory conditions was studied. In FIG. 5, OT-I splenocytes were co-cultured with bone marrow derived Dendritic Cells (DCs) that were treated with LPS and subsequently loaded with either no peptide or peptide OVA (pOVA) (SIINFEKL peptide, specific for the OT-I TCR). Co-cultures were plated in the absence or presence of a saturating amount of IL2 and in the absence or presence of 7D6 Fab. After four days, co-cultures were stained for Thy1.2 and Fas surface expression. Propidium Iodide (PI) was added to the staining procedure to label dead cells, and samples were analyzed by flow cytometry. In FIG. 5A, the counts of T cells alive were determined by analysis of forward and size scatter, in addition to PI exclusion. The results indicate that 7D6 Fab treatment induced the death of T cells independently and dominant to IL2 and antigenic TCR stimulation. The results in FIG. 5B, demonstrate that 7D6 Fab treatment induces the up-regulation of Fas expression on T cells, suggesting that the mechanism of death induced by 7D6 Fab on T cells involves this death receptor.

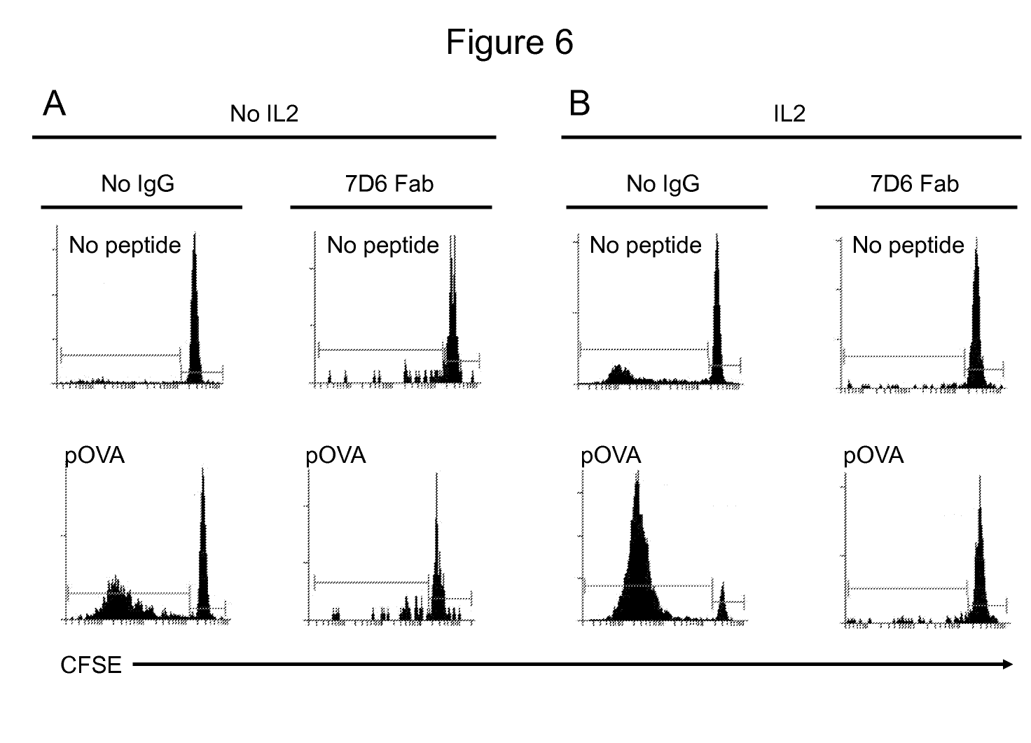

Next, the effect of 7D6 Fab on the cell cycle of T cells was determined. In FIG. 6, CFSE labeled OT-I splenocytes (Carboxyfluorescein succinimidyl ester) co-cultured with DCs. After four days, the CFSE profile of PC Thy1.2⁺ cells was monitored by flow cytometry to account for T cell division induced in the co-cultures. CFSE profiles of co-cultures in the absence (FIG. 6A) or presence (FIG. 6B) of IL2 are shown. These results suggest that 7D6 Fab does not induce T cell division.

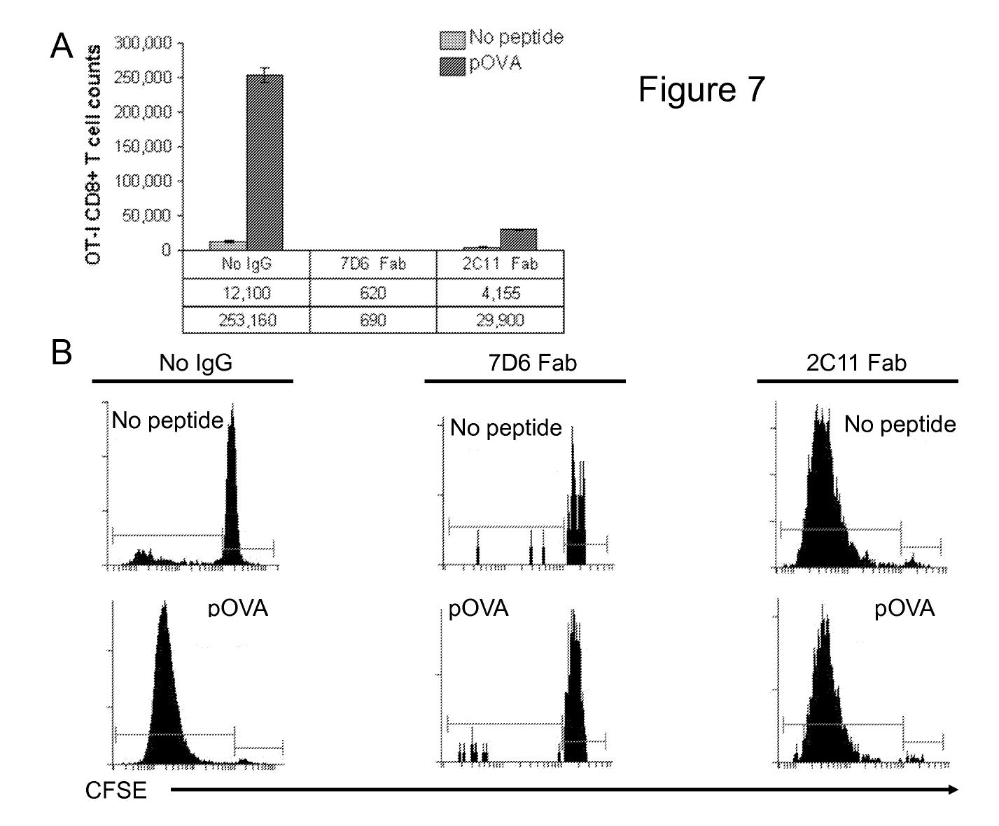

A comparison of the effect of 7D6 Fab with 2C11 Fab was performed; the latter binds to both CD3γε and CD3δε dimers on T cells. CFSE labeled OT-I splenocytes were co-cultured in IL2 with DCs (as in FIG. 5) either in the absence of IgG or the presence of 7D6 Fab or 2C11 Fab. In FIG. 7A, T cell counts present on co-cultures stained for Thy1.2, were determined by analysis of forward and size scatter in addition to PI cell exclusion. In FIG. 7B, CFSE profile of PI⁻ Thy1.2⁺ cells was monitored as well to account for T cell division in the co-cultures. Data from FIGS. 7A and 7B demonstrates that isolated engagement of CD3γε dimers by 7D6 Fab induces the death of T cells without causing T cell division, while co-engagement of CD3γε/εδ dimers by 2C11 Fabs, induces both T cell death and T cell division.

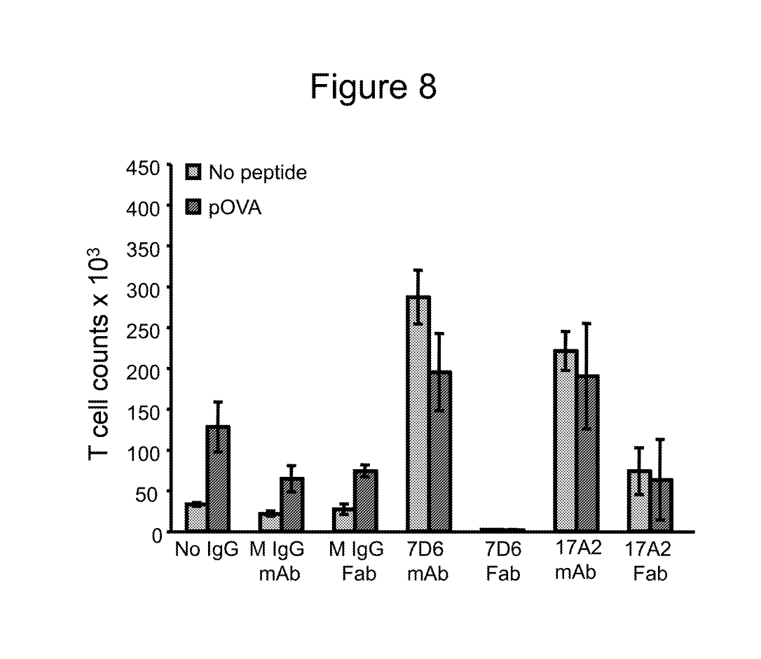

The 7D6 Fab was also compared with an alternative anti-CD3γε Fab, 17A2 Fab. In FIG. 8, OT-I splenocytes were co-cultured in IL2 with DCs (as in FIG. 5), either in the absence of IgG, or the presence of control mouse IgG mAb and Fab, or the presence of anti-CD3γε 7D6 and 17A2 Fabs and mAbs. After four days, co-cultures were stained for Thy1.2, and counts of T cells alive were determined by analysis of forward and size scatter in addition to PI cell exclusion. While 7D6 Fab induces T cell death over basal and stimulatory conditions 17A2 Fab fails to do so. These data indicate that binding to the CD3γε by a given Fab is not enough to produce T cell death without cell division and that 7D6 Fab may possess unique structural and binding properties to support its effects on T cells.

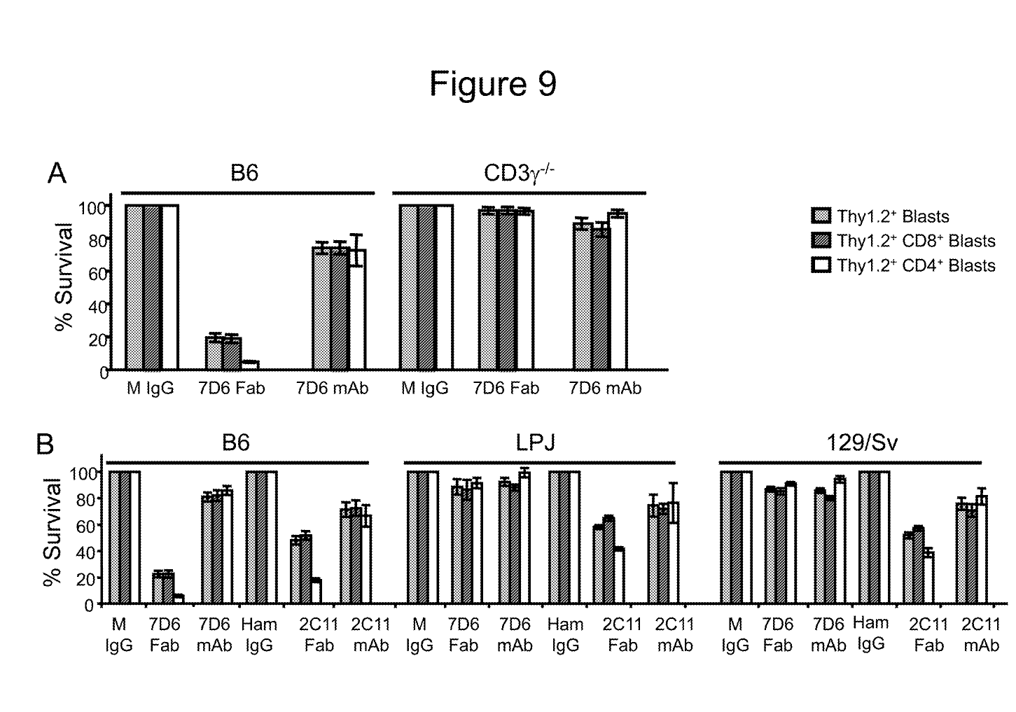

Next, the requirement of 7D6 Fab to bind CD3γε in order to display its effects on T cells was examined First, B6 wild type and CD3γ^{-/-} T cell blasts derived from stimulation of splenocytes for four days with plate bound anti-CD3 were rested for one day and tested for CD3. While B6 blasts expressed normal levels of CD3 on the surface, CD3γ^{-/-} T cell blasts failed to express detectable levels of CD3. Both types of blasts were incubated for 24 hours with either soluble mouse IgG, 7D6 Fab, or 7D6 mAb. Living T cells counts were obtained analyzing forward and size scatter and PI exclusion by flow cytometry, and a percentage of survival of T cell blasts in the presence of 7D6 Fab and mAb was calculated by comparison with the mouse IgG condition (FIG. 9A). Since 7D6 Fab fails to kill CD3γ^{-/-} blasts, it would seem that 7D6 Fab requires the expression of CD3 to cause the death T cells. T cell blasts generated from splenocytes of B6 and the LPJ and 129/Sv strains that do not stain with 7D6 due to allelic variation in CDR were stimulated for 24 hours (as in FIG. 9A), and a percentage survival of T cells blasts in the presence of 7D6 Fab and mAb was calculated (FIG. 9B). Since 7D6 Fab failed to kill all blasts expressing the allelic variants of the CD3γε dimer that fails to bind, it would seem that 7D6 Fab is required to bind to the CD3γε dimer to kill T cells.

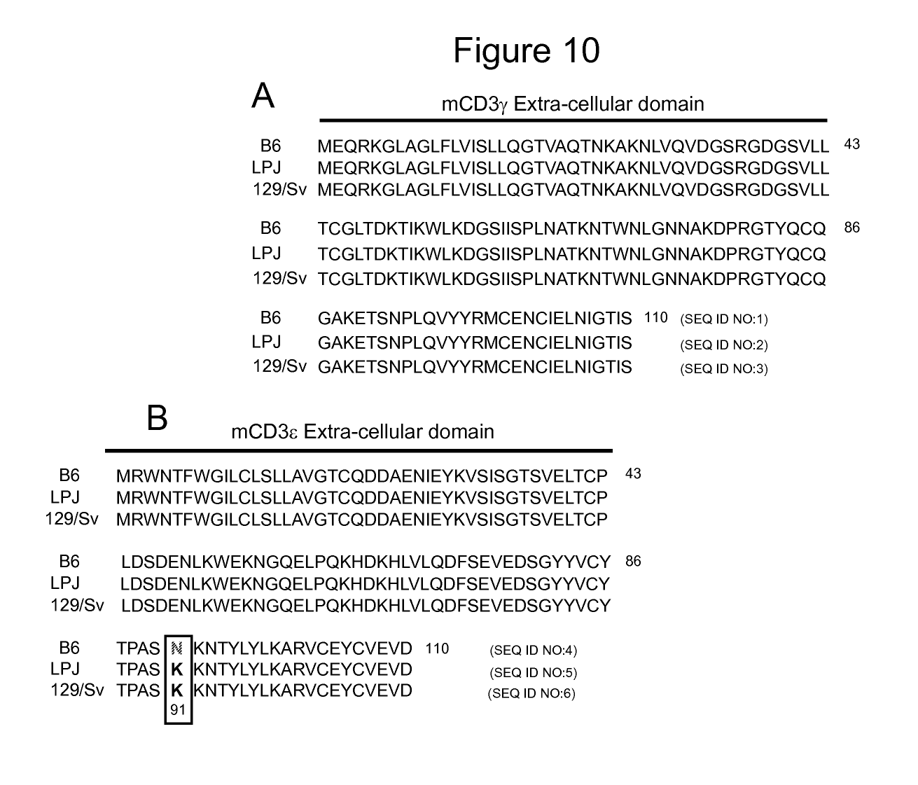

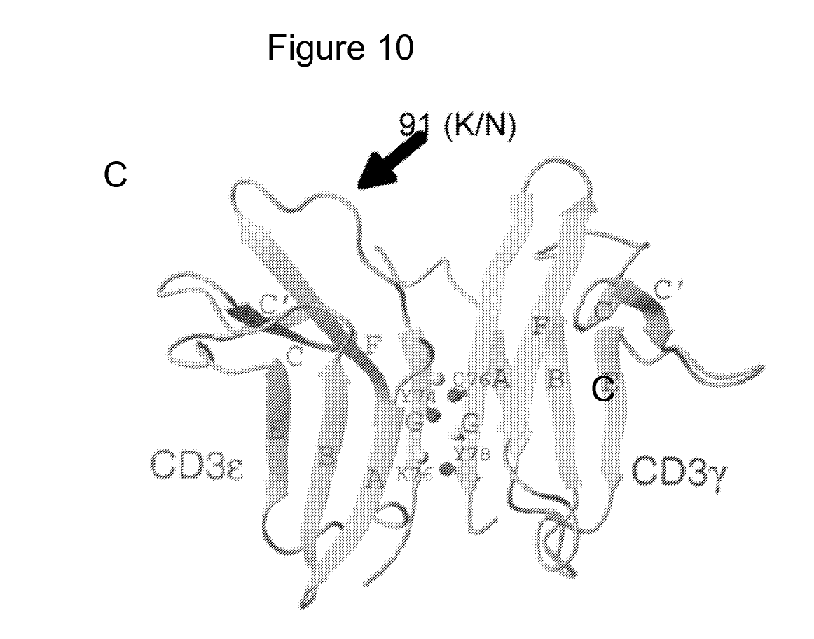

mRNA coding for CD3γ (FIG. 10A) and CDR (FIG. 10B) from the B6, LPJ and 129/Sv mouse strains was sequenced. Resulting sequences were aligned to identify allelic variations. The results demonstrate that position 91 of the extra-cellular domain of CDR in the B6 sequence was changed from N to K in both LPJ and 129/S. This indicates that the epitope recognized by 7D6 in the CD3γε dimer of B6 may include residue 91 of CDR. Based on the location of this residue (FIG. 10C, see arrow) (Sun et al., Cell, 105:913-923 (2001)), it seems that 7D6 epitope may be shaped by residues included in the F/G loops in CD3γ and CDR.

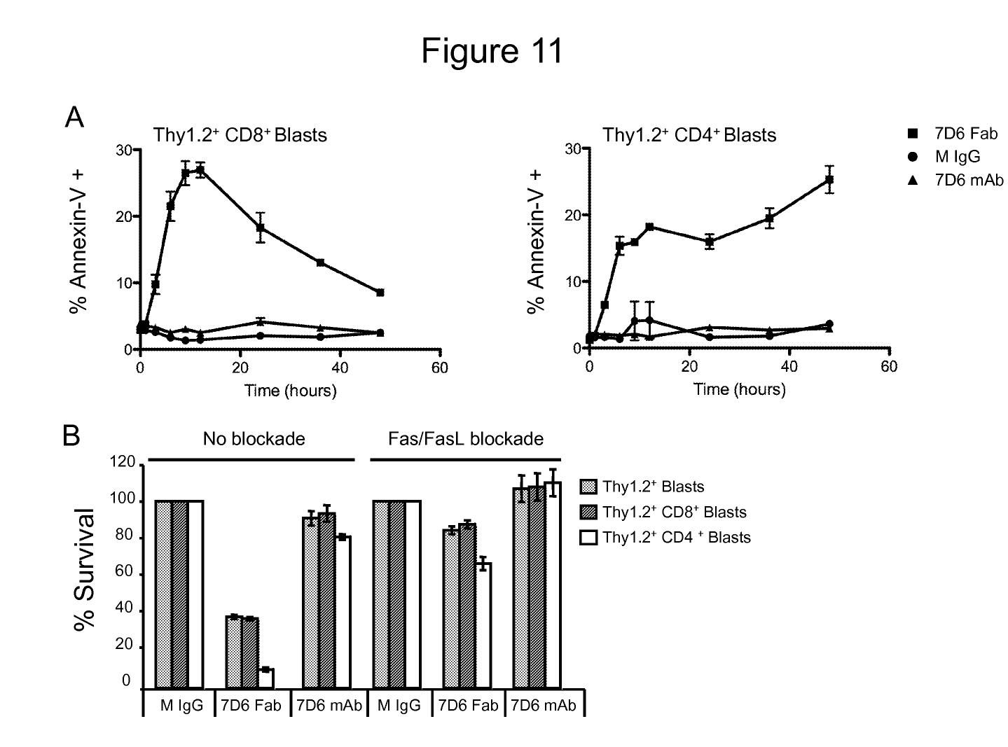

The mechanism utilized by 7D6 Fab to cause the death of T cells was also investigated. Since 7D6 Fab requires binding the CD3γε dimer to kill T cells, it would seem that 7D6 Fab causes T cell apoptosis via Fas/FasL, a mechanism that is activated by stimulation of the TCR/CD3 complex. To test this idea, the induction of apoptosis on T cells by staining with Annexin-V was monitored. B6 T cell blasts derived from stimulation of splenocytes for four days with plate bound anti-CD3 were rested for one day and then incubated with either soluble mouse IgG, 7D6 Fab, or 7D6 mAb. At the time points indicated (FIG. 11A), living CD8 and CD4+ T cell blasts were monitored for Annexin-V staining by flow cytometry, showing both populations increased staining for Annexin-V after 3 hours of incubation with 7D6 Fab. After confirming the induction of apoptosis, the requirement of Fas/FasL signaling for the apoptosis induced by 7D6 Fab was tested. In FIG. 11B, B6 T cell blasts rested for one day, were stimulated for 24 hours with either soluble mouse IgG, 7D6 Fab, or 7D6 mAb, in the presence or absence of a Fas/FasL blocking cocktail. The percentage of survival of T cells blasts was calculated relative to the mouse IgG conditions as in FIG. 9. The inhibition of the death effect by 7D6 Fab in the presence of Fas/FasL blockade indicates that the signaling of Fas/FasL interaction is required by 7D6 Fab to kill T cells.

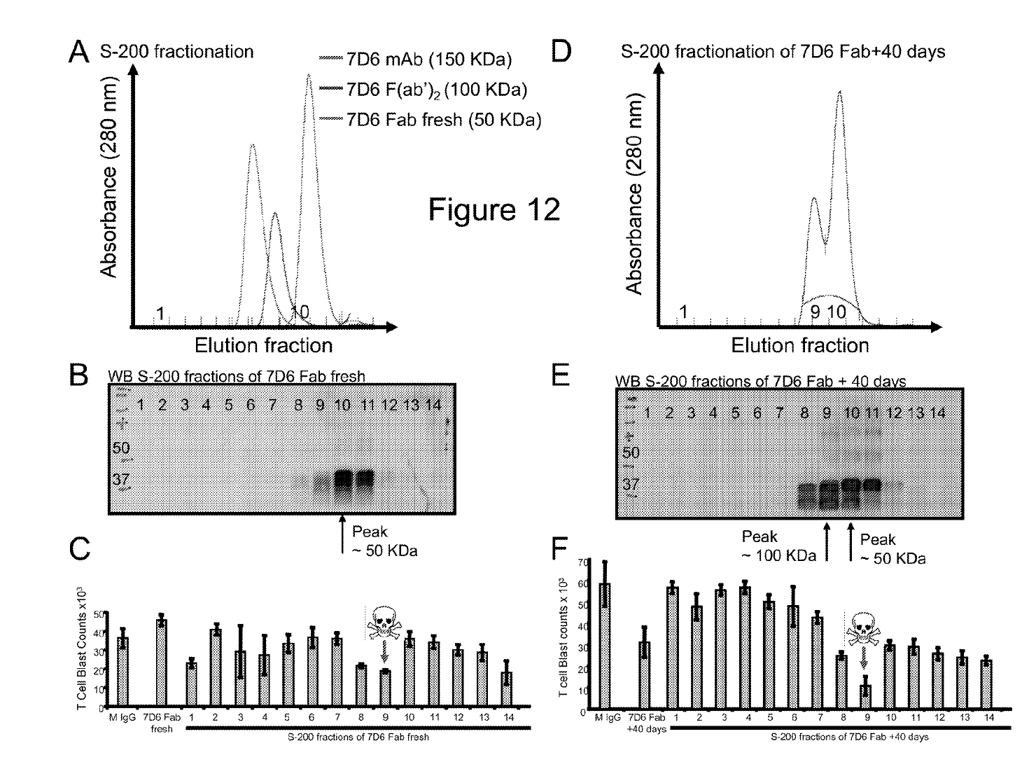

Monovalent engagement of the TCR/CD3 complex is supposed not to trigger CD3 signal transduction and it is expected to fail to induce any T cell responses. However, 7D6 Fab induces CD3 conformational change, on T cells, together with CD69 up-regulation and T cell death, but in the absence of TCR/CD3 internalization or T cell division. This partial pattern of T cell responses indicates that 7D6 Fab is at least partially efficient in triggering CD3 signal transduction and it may not be monovalent as expected for a regular Fab fragment. Therefore, the valency of the 7D6 Fab was studied. FIG. 12A demonstrates the size exclusion chromatography (SEC) profile of 7D6 mAb, 7D6 F(ab′)₂, and 7D6 Fab freshly prepared (7D6 Fab fresh) when fractionated over a Superose-200 column (S-200) equilibrated in PBS. The expected molecular weight for each 7D6 species tested is shown in brackets. In FIG. 12B, 7D6 Fab fresh fractions collected in 12A were run in SDS-PAGE non-reducing conditions to detect 7D6 by anti-mouse western blotting. The peak of maximum 7D6 Fab fresh material is detected in fraction 10, which matches the SEC elution peak of 7D6 Fab fresh in 12A. Moreover the 7D6 Fab fresh band detected by western blot displays the expected molecular size around 50 KDa. These results indicate that 7D6 Fab freshly prepared is monovalent. Further experiments were performed to determine whether 7D6 Fab fresh could kill T cells. FIG. 12C contains results from a killing activity test against T cell blasts performed as in FIG. 9 with the 14 fractions of 7D6 Fab fresh collected in 12A. Note that unfractionated 7D6 Fab fresh does not display a significant killing activity over the T cell blast tested. However, a peak of killing activity against T cells was found in fraction 9 of the 7D6 Fab fresh. Note that fraction 9 is where molecular species of 100 KDa elute, as shown for the 7D6 F(ab′)₂ in FIG. 12A. This result indicates the presence of a sub-species in 7D6 Fab fresh of 100 KDa that displays killing activity against T cells when isolated by SEC fractionation. This sub-species could represent the spontaneous dimerization of monomeric 7D6 Fab molecules that accumulates with time. To test this hypothesis, the SEC profile of 7D6 Fab 40 days after its preparation (7D6 Fab aged) was studied. As shown in FIG. 12D, 7D6 Fab aged displays an additional elution peak in fraction 9. Fractions collected in 12D were run in SDS-PAGE non-reducing conditions in 12E to detect 7D6 by anti-mouse western blotting. In this case, the maximum amount of 7D6 Fab aged spreads between fractions 9 and 10, matching the two SEC elution peaks shown in 12D. Note as well that some of the 7D6 Fab aged monomers seem to have decreased in size when compared with results from 12C. These observations suggest that monomeric 7D6 Fab molecules (7D6-Mono-Fab) form non-covalent associations of 100 KDa with time. Given the size reduction of some of the monomers in the 7D6 Fab aged, the 100 KDa 7D6 Fab species (7D6-Oligo-Fab) may contain from 2 to 4 molecules of 7D6-Mono-Fab.

Finally, the killing properties of 7D6 Fab aged fractions were studied. FIG. 12F shows that fraction 9 contains the peak of maximum killing activity against T cells. These data indicated that 7D6-Oligo-Fab molecules of 100 KDa are responsible of the killing of T cell blasts seen in these experiments.

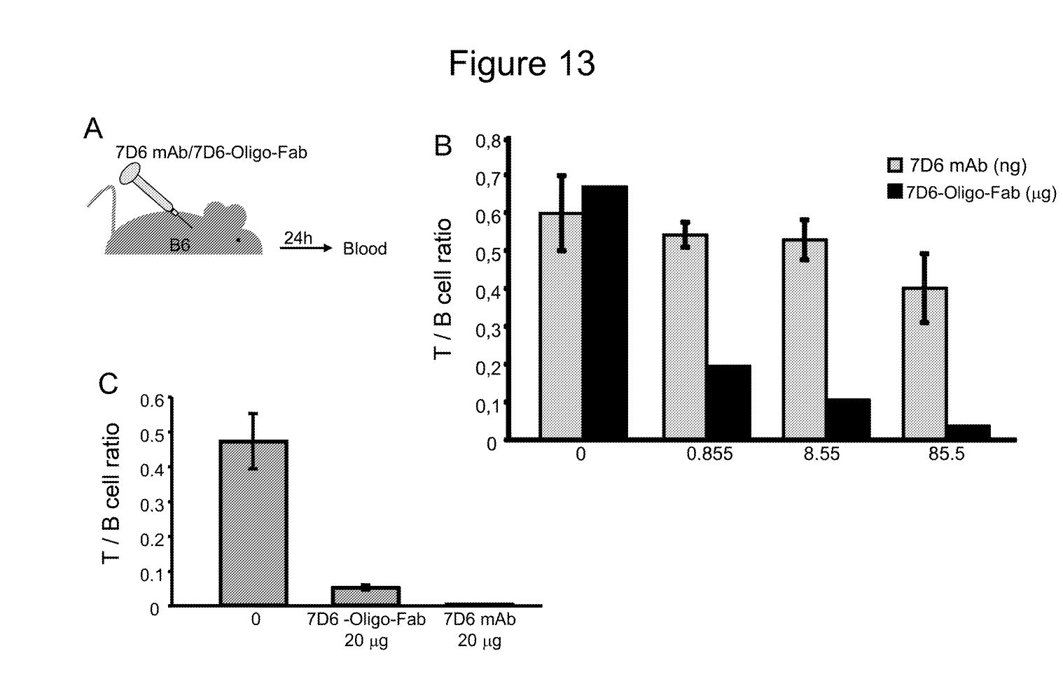

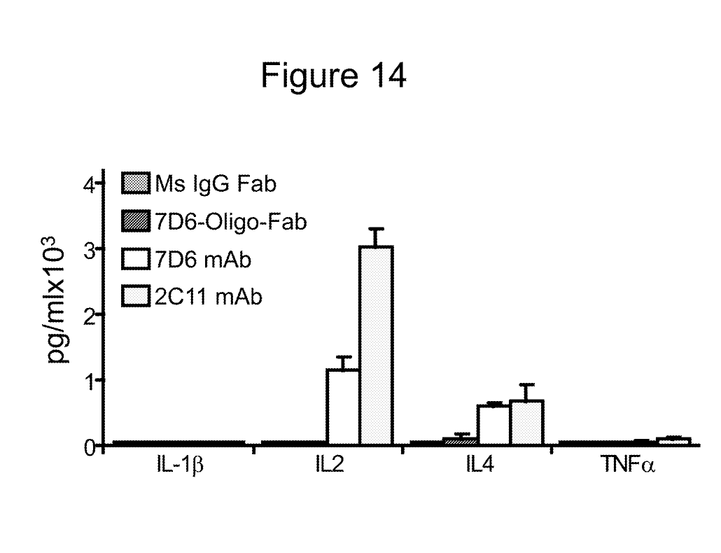

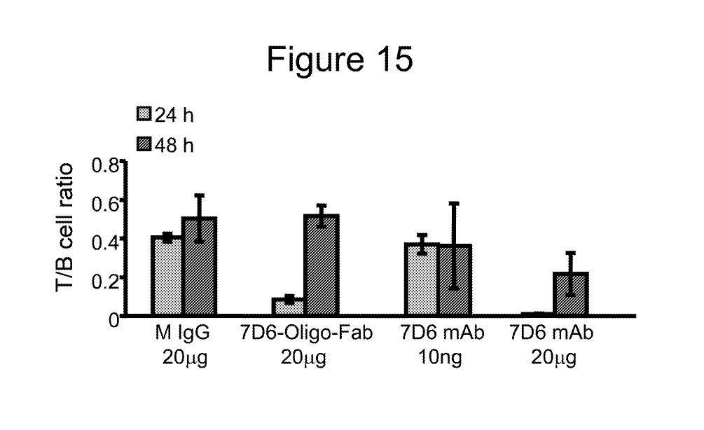

Given the capacity of 7D6 Fab to kill T cells without causing T cell division, the consequences of injecting 7D6-Oligo-Fab in mice were further studied to determine the possibility of using this reagent to deplete T cells in vivo without provoking the adverse effects following T cell expansion and cytokine production. Intraperitoneal (i.p.) injection of 7D6-Oligo-Fab into B6 mice was performed as depicted in FIG. 13A. Three B6 mice per experimental group were injected with the indicated doses of 7D6 mAb (as a control for the presence of residual undigested 7D6 mAb in 7D6 Fab preparations) and 7D6 Fab as indicated in FIG. 13B, and PBLs were isolated from blood. The expression of B2.20 and Thy1.2 surface markers on the PBLs was monitored by flow cytometry, and a T to B cell ratio was calculated in each sample based on the results of the stainings. Data shown from this experiment in FIG. 13B indicates that a significant depletion of T cells over the contamination control of undigested 7D6 mAb is achieved with a dose range from 0.85 to 85 μg/mouse of 7D6 Fab. In subsequent experiments, a working dose of 20 mg/mouse as shown in FIGS. 13C and 15 was established. Once an effective dose to deplete T cells was established, experiments were performed to study whether 7D6-Oligo-Fab may cause cytokine release syndrome (CRS). In FIG. 14, three B6 mice per experimental group were injected intravenously with 20 μg of the non-specific mouse IgG Fab, 7D6-Oligo-Fab, 7D6 mAb, or 2C11 mAb. Two hours later, blood samples were collected, and sera were prepared and analyzed for the specified cytokine concentrations by ELISA. As shown in FIG. 14, 7D6-Oligo-Fab did not promote the secretion of any of the cytokines measured in the experiment (IL-1β, IL-2, IL-4, TNF-α), while 7D6 and 2C11 mAbs stimulated secretion of IL-2 and IL-4. Mice involved in the experiment depicted in FIG. 14 were also monitored for CRS symptoms as piloerection, diarrhea, and hypoglycimia, testing negative for all related measurements. These observations indicated that 7D6-Oligo-Fab is suitable to cause T cell depletion from blood without causing adverse side effects related with CRS.

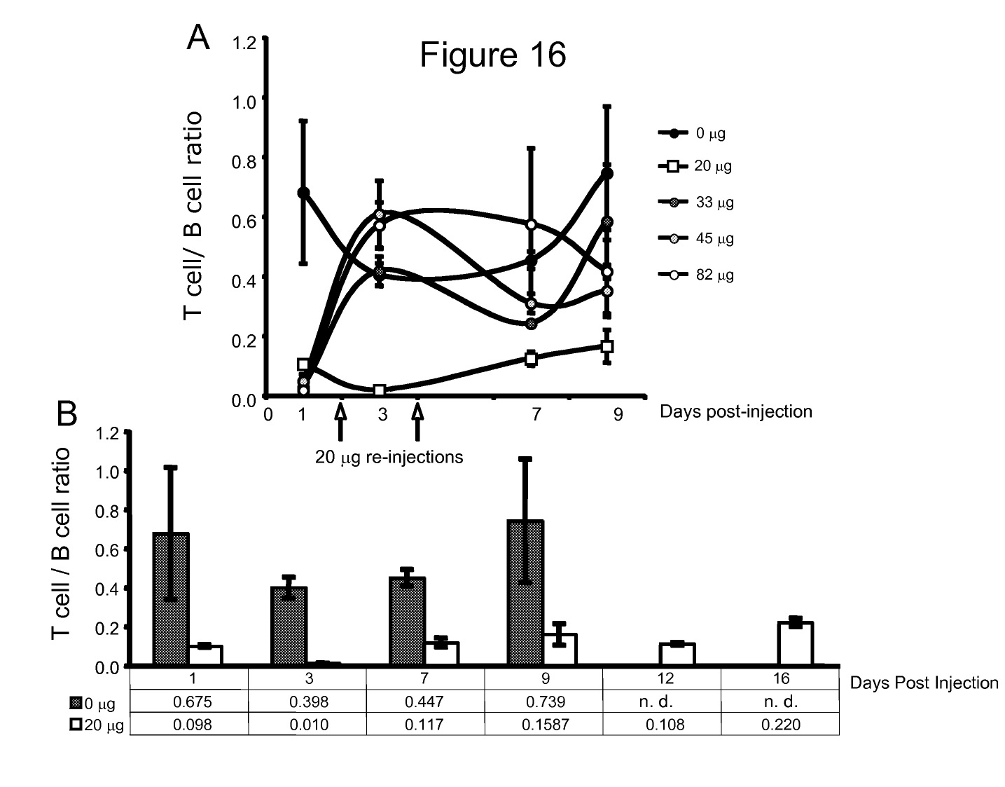

Experiments were performed to establish the kinetics of the T cell depletion from blood caused by 7D6-Oligo-Fab. Three B6 mice per experimental group were injected retro-orbitally with the indicated doses in FIG. 15 for mouse IgG control and different 7D6 Abs. 24 and 48 hours post-injection, mice were bled, and PBLs were isolated. Surface expression of the B cell marker B2.20 and the T cell marker Thy1.2 was monitored in the PBLs by flow cytometry, and a T to B cell ratio was calculated in each sample based on the results of the B2.20 and Thy1.2 stainings. Results from FIG. 15 indicate that a single injection of 7D6-Oligo-Fab induced just a transitory depletion, since T cell numbers recovered normal levels 48 hours after the injection. Interested in achieving a sustained T cell depletion, the effects of either increasing the dose of 7D6-Oligo-Fab injected or repeating the injection of 20 μg of 7D6-Oligo-Fab into mice were explored. Three B6 mice per experimental group were injected with the indicated doses of 7D6-Oligo-Fab as in FIG. 16A. Mice receiving a 20 μg dose were re-injected on day 2 and 4 after their first injection. At the indicated time points, mice were bled, and PBLs were isolated from blood. The expression of B2.20 and Thy1.2 surface markers on the PBLs was monitored by flow cytometry, and a T to B cell ratio was calculated. Data in FIG. 16A shows that repeated injections of 20 μg of 7D6-Oligo-Fab are more effective at maintaining T cell depletion in blood than single injections of higher doses of 7D6-Oligo-Fab. FIG. 16B shows that depletion achieved by the three injections of 20 μg of 7D6-Oligo-Fab was sustained for 12 days and started to recover at 16 days.

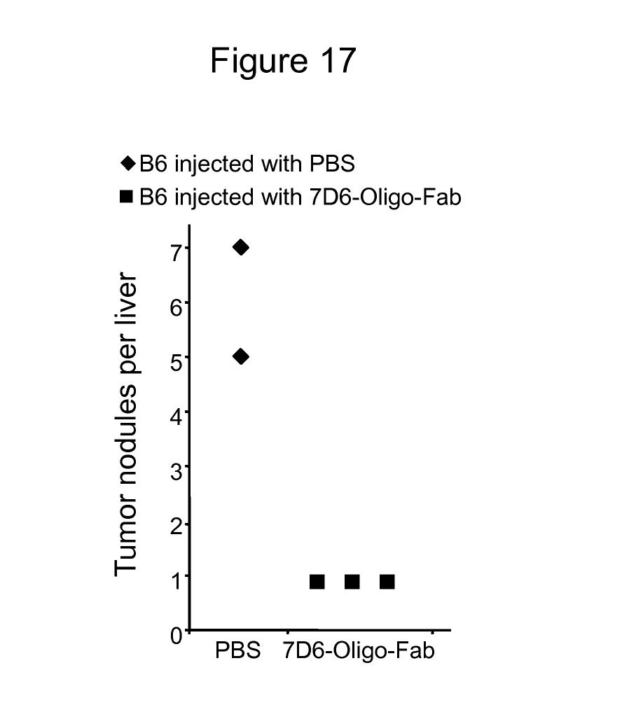

Since repeated injections of 7D6-Oligo-Fab maintained low levels of T cell in blood without causing deleterious side effects, the administration of 7D6-Oligo-Fab into mice with pathologies related with T cell function was tested and amelioration and/or delay of disease course was examined First, 7D6-Oligo-Fab was administered into a new murine model of T cell lymphoma. The Tot1.1 cell line was derived upon extensive in vitro culture of a spontaneous T cell lymphoma that occurs in a B6 OT-I mouse colony. Five cage-mate B6 mice were injected i.v. with 0.5×10⁶ Tot1.1 tumor cells. Six days later, two mice were injected i.p. with PBS, while three mice were injected i.p. with 20 μg of mono-7D6-Fab. After 17 days, mice injected with PBS showed significant signs of morbidity, but those injected with mono-7D6-Fab did not. All mice were sacrificed on day 17, and putative tumor-containing nodules were harvested from livers. Each putative tumor-containing nodule was placed in its own tissue culture well for one week. Nodules were verified as tumor-positive if Tot1.1 cells grew from them in culture. Results from this experiment are depicted in FIG. 17. It was found that 7D6-Oligo-Fab reduced Tot1.1 tumor burden in mice, suggesting that the killing activity of this Fab acted against tumor progression.

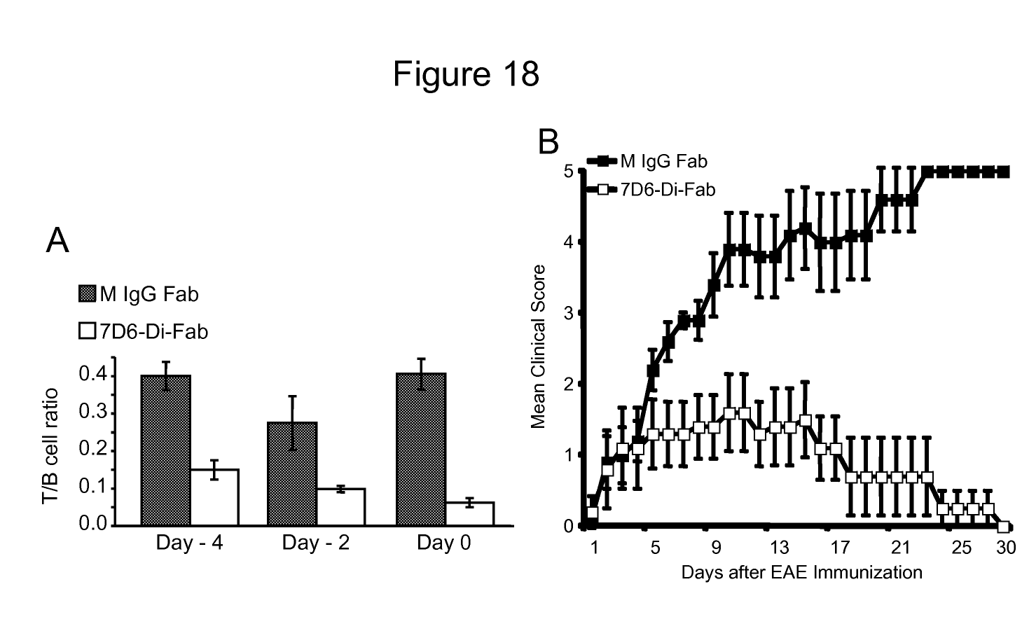

Next, the effect of 7D6-Oligo-Fab administration into a mouse model for multiple sclerosis, a T cell driven autoimmune disease that attacks the central nervous system (CNS), was explored. In this model, B6 mice develop experimental autoimmune encephalomyelitis (EAE) when an aggressive immunization regimen with a peptide derived from a myelin protein allows T cells to cross the blood barrier and reach the CNS. B6 mice were retro-orbitally injected with either 20 μg of mouse IgG or 7D6-Oligo-Fab (5 mice/condition) at days −5, −3 and −1. 24 hours post-Fab injections, mice were bled, and PBLs were isolated from blood. The expression of B2.20 and Thy1.2 surface markers on the PBLs was monitored by flow cytometry. A T to B cell ratio was calculated showing significant depletion of T cells by 7D6-Oligo-Fab treatment by day 0. FIG. 18A shows the depletion of T cells from the blood caused by 7D6-Oligo-Fab injections. At day 0, the immunization protocol to cause EAE in B6 was applied to all mice engaged in the experiment. Mice were immunized s.c. in both flanks with 100 μg of the peptide MOG emulsified in CFA containing M. tuberculosis H37Ra (400 μg/mice), and Pertussis toxin (100 ng) was injected i.v. Forty-eight hours later, Pertussis toxin was re-injected to all mice. Mice were observed daily for clinical symptoms of EAE and disease severity was scored as follows: 0, normal; 1, loss of tail tone; 2, hind limb weakness; 3, hind limb paralysis; 4, hind limb paralysis and forelimb paralysis or weakness; and 5, moribundity/death/mouse was sacrificed. In FIG. 18B, a mean clinical disease score is depicted. By day 47 all mice from the 7D6-Oligo-Fab experimental group were recovered from EAE and scored as 0, and remained symptom-free for as long as they were monitored, until at least day 77. These data indicated that 7D6-Oligo-Fab administration right before the immunization regimen first causes de amelioration of the disease course, since mice develop disease with same kinetics as controls but with lower clinical score, and second promotes full recovery of afflicted mice.

Taken together, the results presented herein indicate that anti-CD3γε antibody preparations can be used to induce therapeutic T cell death in vivo in mammals without causing the unwanted side-effects of either T cell proliferation or cytokine production and may as well invoke other pro-tolerance mechanisms that protect against autoimmune diseases.

### Example 2

### Producing Humanized Anti-Human CD3γε Antibodies

A first nucleic acid construct is constructed to encode the extracellular domain of human CD3εfused to the transmembrane and cytoplasmic domains of mouse CD3ε, while a second nucleic acid construct is constructed to encode the extracellular domain of human CD3γ fused to the transmembrane and cytoplasmic domains of mouse CD3γ. The first and second nucleic acid constructs are engineered into a vector to produce a multicistronic vector. A transfection technique such as a retroviral transduction is used to introduce the multicistronic vector into mouse T cells such that the mouse T cells are capable of expressing the human/mouse chimeric CD3ε polypeptide and the human/mouse chimeric CD3γ polypeptide. A monoclonal antibody specific for the human CD3εextracellular domain (e.g., OKT3) is used to test for proper surface expression of the chimeric human/mouse CD3γε dimer.

Mouse T cells expressing the chimeric human/mouse CD3γε dimer are adoptively transferred into a syngeneic mouse recipient under conventional conditions of immunization to trigger an immune response against the human components of the chimeric CD3γε dimer expressed by the transplanted T cells. Conventional protocols are used to generate monoclonal hybridomas from the fusion of splenocytes of the immunized mice with appropriate fusion partners. Monoclonal antibodies specific for the human CD3γε extracellular domain are identified by the positive staining of either (a) the mouse T cells expressing the chimeric human/mouse CD3γε dimer or (b) human T cells, with a supernatant of a cloned hybridoma.

A Fab fragment preparation is prepared by treating an identified positive monoclonal antibody with papain.

Naïve and activated human T cells are isolated from peripheral blood and are treated with non-fractionated and size exclusion fractionated Fab fragments. The resulting T cell responses are evaluated in vitro to determine properties of the Fab fragments. In addition, Fab fragment preparations are administered to humanized mouse models to determine in vivo properties of the Fab fragments. Fab fragment preparations having the ability to deplete human T cells without causing T cell division or cytokine production are selected. The selected antibodies are humanized as described elsewhere (Jones et al., Nature, 321:522 (1986); Riechmann et al., Nature, 332:323 (1988); Verhoeyen et al., Science, 239:1534 (1988); Carter et al., Proc. Nat'l. Acad. Sci. USA, 89:4285 (1992); Sandhu, Grit. Rev. Biotech., 12:437 (1992); and Singer et al., J. Immunol., 150:2844 (1993)). Fab preparations of resulting humanized antibodies are evaluated to confirm that the humanized antibody remains capable of depleting human T cells without causing detrimental T cell division or cytokine production.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An anti-CD3γε antibody preparation comprising Fab fragments of an anti-CD3γε antibody.

2. The preparation of claim 1, wherein said Fab fragments are Fab fragments of an anti-human CD3γε antibody.

3. The preparation of claim 1, wherein said Fab fragments are Fab fragments of a humanized anti-human CD3γε antibody.

4. The preparation of claim 1, wherein said Fab fragments are Fab fragments of a fully human anti-human CD3γε antibody.

5. The preparation of claim 1, wherein administration of said preparation to a mammal induces T cell death within said mammal.

6. The preparation of claim 1, wherein administration of said preparation to a mammal induces T cell death within said mammal with no detectable T cell division.

7. The preparation of claim 1, wherein administration of said preparation to a mammal induces T cell death within said mammal with no detectable increases in IL-1β production.

8. The preparation of claim 1, wherein administration of said preparation to a mammal induces T cell death within said mammal with no detectable increases in IL-2 production.

9. The preparation of claim 1, wherein administration of said preparation to a mammal induces T cell death within said mammal with no detectable increases in IL-4 production.

10. The preparation of claim 1, wherein administration of said preparation to a mammal induces T cell death within said mammal with no detectable increases in TNF-α production.

11. The preparation of claim 1, wherein administration of said preparation to a mammal induces T cell death within said mammal with no detectable increases in IL-1β, IL-2, IL-4, and TNF-α production.

12. A method for reducing the likelihood of transplant rejection in a mammal, wherein said method comprising administering an anti-CD3γε antibody preparation comprising Fab fragments of an anti-CD3γε antibody to said mammal under conditions wherein at least a portion of T cells present within said mammal are killed with no detectable increase in IL-1β, IL-2, IL-4, or TNF-α production.

13. The method of claim 12, wherein said mammal is a human, and said Fab fragments are Fab fragments of an anti-human CD3γε antibody.

14. The method of claim 12, wherein said Fab fragments are Fab fragments of a humanized anti-human CD3γε antibody.

15. The method of claim 12, wherein said Fab fragments are Fab fragments of a fully human anti-human CD3γε antibody.

16. A method for treating a mammal having an autoimmune condition or cancer, wherein said method comprising administering an anti-CD3γε antibody preparation comprising Fab fragments of an anti-CD3γε antibody to said mammal under conditions wherein at least a portion of T cells present within said mammal are killed with no detectable increase in IL-1β, IL-2, IL-4, or TNF-α production.

17. The method of claim 16, wherein said mammal is a human, and said Fab fragments are Fab fragments of an anti-human CD3γε antibody.

18. The method of claim 16, wherein said Fab fragments are Fab fragments of a humanized anti-human CD3γε antibody.

19. The method of claim 16, wherein said Fab fragments are Fab fragments of a fully human anti-human CD3γε antibody.

20. The method of claim 16, wherein said autoimmune condition is diabetes or multiple sclerosis.

21-25. (canceled)
